(19) United States
(12) Patent Application Publication
(10) Pub. No.: US 2017/0020600 A1
(43) Pub. Date: Jan. 26, 2017

(54) ROTATION STRUCTURE OF ELECTROSURGICAL PEN

(71) Applicant: SHINING WORLD HEALTH CARE CO., LTD., New Taipei City (TW)

(72) Inventor: Su-Chu Lien, New Taipei City (TW)

(21) Appl. No.: 14/805,473

(22) Filed: Jul. 22, 2015

(51) Int. Cl. A61B 18/14; H01R 12/75

(52) U.S. Cl. CPC A61B 18/148; H01R 12/75; A61B 2018/00202

(57) ABSTRACT

A rotation structure of an electrosurgical pen includes a pen body and a cutting tip mounted to the pen body, an extendible/retractable smoke suction tube, and a rotation assembly mounted to an end of the pen body, and a conduit rotatably coupled to the rotation assembly. The pen body has an end on which a rotary joint part is formed. The rotation assembly includes inner and outer annular members that are rotatable with respect to each other. The rotation assembly is fit over and rotatably coupled to the rotary joint part. The conduit is fit over an outer circumference of the outer annular member. In a use of the electrosurgical pen, the rotation assembly allows the smoke suction tube and the conduit to rotate relative to each other so as to allow for free rotation of the electrosurgical pen to facilitate the operation of the electrosurgical pen.

### (a) TECHNICAL FIELD OF THE INVENTION

The present invention relates generally to a rotation structure of an electrosurgical pen, and more particularly to a rotation structure that allows for free rotation of an electrosurgical pen in order to facilitate the operation of the electrosurgical pen.

### (b) DESCRIPTION OF THE PRIOR ART

Surgical operations are serious and difficult medical treatments and were heretofore conducted with surgical blades, with which a common problem to be handled is bleeding issues. With the progress and advance of science technology, nowadays, surgical operations may be conducted with electrosurgery, which is gradually accepted by doctors and patients. Electrosurgery uses an electrical current having a waveform frequency of 100 KHz-10 MHZ that is conducted through human body to have human body heating through resistance of the human body in order to damage the tissue of the affected part and achieve an effect of fast cutting or fast coagulation. This helps achieve the purpose of reduce bleeding in a surgical operation. During an electrosurgical operation, smokes may be generated and must be removed with a smoke suction tube to prevent undesired interference with the performance of the operation. Thus, an electrosurgical unit or electrosurgical pen must be coupled to at least a conduit and electrical wires received in and extending through the conduit. The electrical wires and conduit coupled to the electrosurgical pen constrains the movement of the electrosurgical pen and prevents the electrosurgical pen from being rotated freely. As such, the use and operation of the electrosurgical pen is inconvenient and the performance of a surgical operation may be interfered with. The present invention aims to provide a rotation structure of an electrosurgical pen that allows for easy and flexible operation of the electrosurgical pen.

### SUMMARY OF THE INVENTION

The primary object of the present invention is to provide a rotation structure of an electrosurgical pen, which allows for free rotation of the electrosurgical pen and eases the operation of the electrosurgical pen.

The rotation structure of the electrosurgical pen comprises a pen body and a cutting tip mounted to the pen body, an extendible/retractable smoke suction tube, and a rotation assembly mounted to an end of the pen body, and a conduit rotatably coupled to the rotation assembly. The pen body has an end on which a rotary joint part is formed. The rotation assembly comprises inner and outer annular members that are rotatable with respect to each other. The rotation assembly is fit over and rotatably coupled to the rotary joint part of the pen body. The conduit is fit over an outer circumference of the outer annular member of the rotation assembly. In a use of the electrosurgical pen, the rotation assembly allows the smoke suction tube and the conduit to rotate relative to each other so as to allow for free rotation of the electrosurgical pen to facilitate the operation of the electrosurgical pen.

In the above rotation structure of the electrosurgical pen, the rotary joint part of the pen body comprises a flange formed on the end of the pen body, an insertion section extending from the flange, and a diameter-expanded retention section extending from the insertion section to receive the rotation assembly to be fit over the insertion section and retained in position by the retention section so as to prevent detachment of the rotation assembly.

In the above rotation structure of the electrosurgical pen, the insertion section and the retention section of the rotary joint part of the end of the pen body comprise a plurality of slits formed therein to provide elasticity that facilitates fitting of the rotation assembly.

In the above rotation structure of the electrosurgical pen, the rotation assembly comprises a bearing.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the present invention.

FIG. 2 is a perspective view illustrating the structure of the present invention.

FIG. 3 is an exploded view showing a rotation structure of the present invention.

FIG. 4 is a cross-sectional view showing the rotation structure of the present invention.

FIG. 5 is a schematic view illustrating an application of the present invention.

FIG. 6 is a schematic view illustrating a condition of operation of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

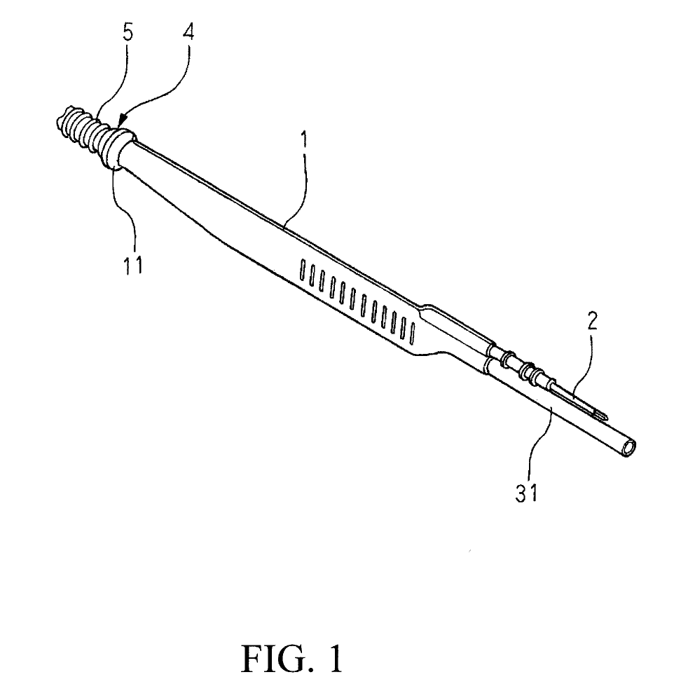

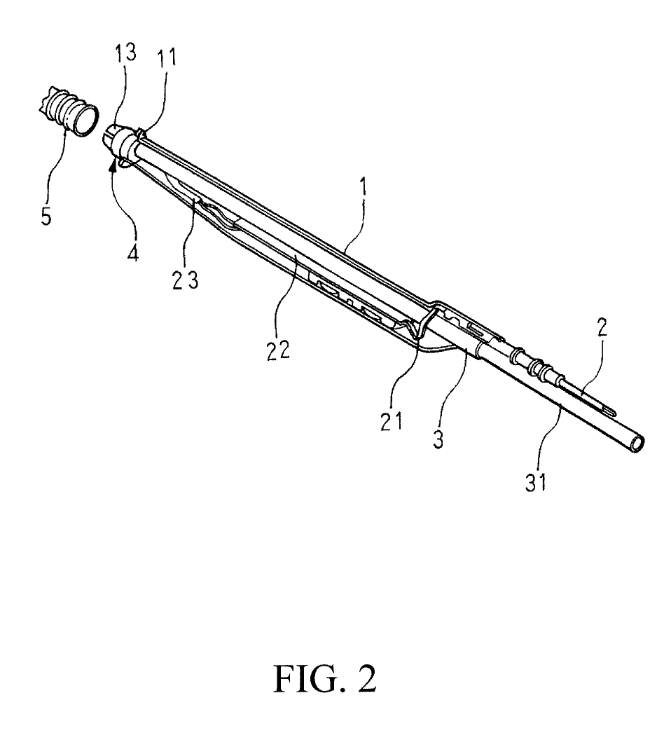

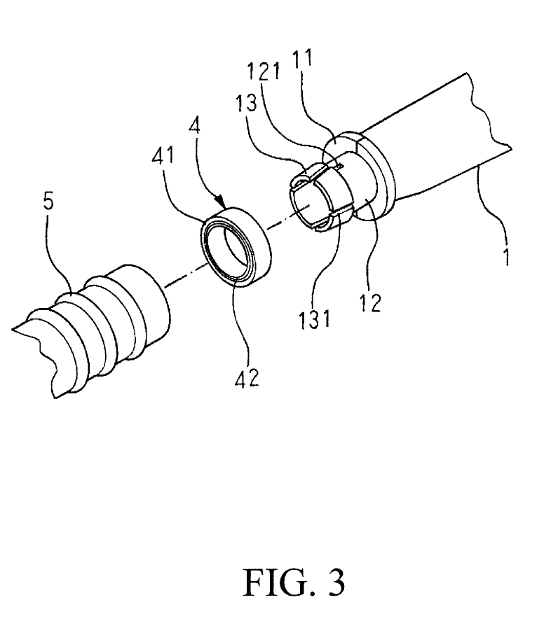

Referring to FIGS. 1 and 2, in combination with FIG. 3, which are respectively a perspective view of the present invention, a perspective view illustrating the structure of the present invention, and an exploded view of a rotation structure of the present invention, as shown in the drawings, the present invention comprises a pen body 1 and a cutting tip 2 and a smoke suction tube 3 mounted to the pen body 1, a rotation assembly 4 coupled to the pen body 1, and a conduit 5 connected to the rotation assembly 4. The pen body 1 has an end on which a rotary joint part is formed. The rotary joint part comprises a flange 11 formed on the end of the pen body 1, an insertion section 12 extending from the flange 11, and a diameter-expanded retention section 13 extending from the insertion section 12. The insertion section 12 and the retention section 13 comprise a plurality of slits 121, 131, which has open ends in an end of the retention section, to provide elasticity.

The cutting tip 2 is mounted on a top of the pen body 1 and projects outwards from a front end of the pen body 1 and is connected, via a conductive plate 21, to a circuit board 22 to be further connected by the circuit board 22 to an electrical cable 23. The electrical cable 23 is received in and extends through the smoke suction tube 3 and the conduit 5.

The smoke suction tube 3 comprises an inner tube 31. The inner tube 31 is exposed outside and projects frontwards from the front end of the pen body 1 to be extendible and retractable with respect to out of the smoke suction tube 3 to accommodate the cutting tip 2.

The rotation assembly 4 comprises an outer annular member 41 and the inner annular member 42 that are rotatable with respect to each other and the inner annular member 42 is fit over the insertion section 12 of the rotary joint part of the end of the pen body 1 so as to be retained in position between the flange 11 and the retention section 13 to allow the conduit 5 to fit over an outer circumference of the outer annular member 41 of the rotation assembly 4, allowing the pen body 1 to rotate about the rotation assembly 4 to allowing the conduit 5 to rotate about the rotation assembly 4 so as to achieve relative rotation between the pen body 1 and the conduit 5.

With the combination of the above-discussed components, an electrosurgical pen is provided for surgical operations, in which the electrical cable 23 is connectable to an electrical power device and the conduit 5 is connectable to a smoke exhausting device. When the electrosurgical pen is used to conduct a surgical operation, the cutting tip 2 that is supplied with electrical power can be used to for electrosurgery. The smoke suction tube 3 may suck and exhaust the smoke generated during the electrosurgical operation. During the operation, the rotation assembly 4 allows the pen body 1 and the conduit 5 to rotate with respect to each other so as to allow for free rotation of the electrosurgical pen to facilitate the operation of the electrosurgical pen.

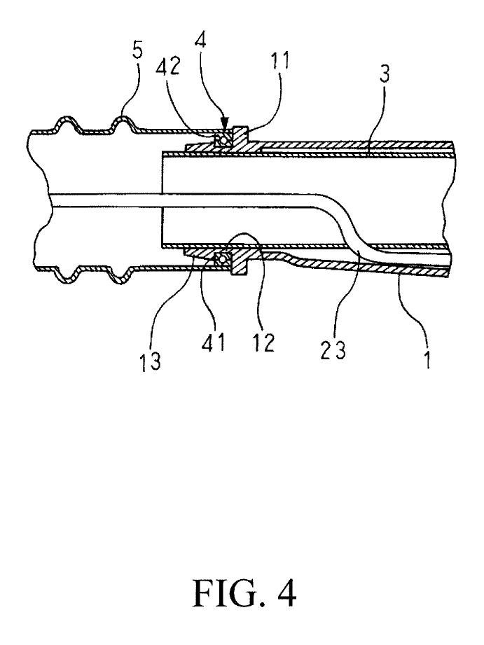

Referring to FIG. 4, an exploded view of the rotation structure of the present invention is illustrated. As shown in the drawing, the insertion section 12 of the pen body 1 is fit into and coupled to the inner annular member 42 of the rotation assembly 4 with retention being achieved with and between the flange 11 and the retention section 13 of the pen body 1. The conduit 5 is fit over and coupled to the outer circumference of the outer annular member 41 of the rotation assembly 4. As such, air tightness may be achieved between the pen body 1 and the conduit 4 and the pen body 1 is freely rotatable bout the rotation assembly 4 or the conduit 5 is freely rotatable about the rotation assembly 4 to achieve relative rotation between the pen body 1 and the conduit 4. This allows for free rotation of the pen body 1 to follow desired directions required for an operation.

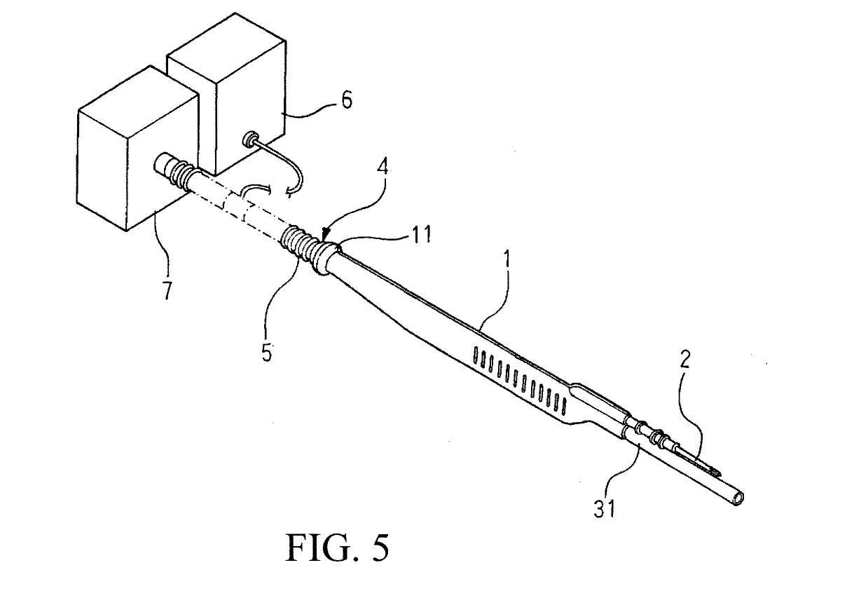

Referring to FIG. 5, a schematic view is given to illustrate an application of the present invention. Reference being also had to FIG. 2, as shown in the drawings, to use the present invention, the electrical cable 23 is connected to an electrical power device 6 and the conduit 5 is connected to a smoke exhausting device 7. The electrical power device 6 supplies electrical power necessary for the circuit board 22 and allows electricity to be conducted to the cutting tip 2 to carry out an electrosurgical operation. The inner tube 31 of the smoke suction tube 3 is allowed to extend outward or inward retract by a proper amount to accommodate the arrangement of the cutting tip 2 so that smoke generated during the electrosurgical operation can be exhausted and removed by the smoke exhausting device 7 through the smoke suction tube 3 and the conduit 5 thereby avoiding interference with the electrosurgical operation caused by the smoke and also helping prevent inhalation of (or even infected by) the smoke by the medical personnel during the operation.

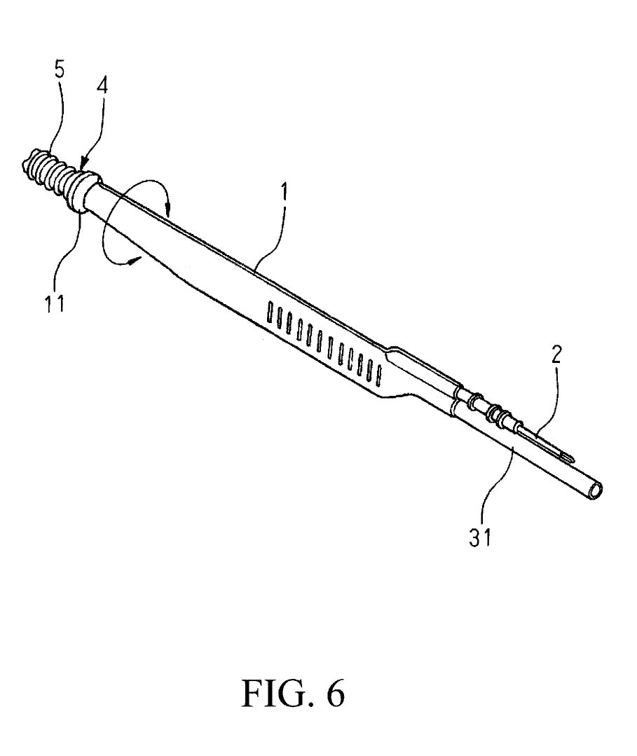

Referring to FIG. 6, a schematic view is given to illustrate a condition of operation of the present invention. Reference being also had to FIGS. 3 and 4, as shown in the drawings, when the electrosurgical pen of the present invention is used in a surgical operation, during the operation, it may need to change the direction and angle of the pen, the present invention allows for the movement or rotation of the electrosurgical pen by providing the rotation structure that allows the pen body 1 to rotate about the rotation assembly 4 or allows the conduit 5 to rotate about the rotation assembly 4 so that the pen body 1 and the conduit 5 are rotatable with respect to each other to facilitate the surgical operation to be conducted with the free rotation of the electrosurgical pen and also to facilitate the use of the electrosurgical pen, making the surgical operation performed in a more smooth manner.

In summary, the present invention provides a rotation assembly that comprises an inner annular member and an outer annular member that are rotatable with respect to each other and a smoke suction tube and a conduit of the electrosurgical pen are respectively coupled to the inner and outer annular members to form a rotation structure of the electrosurgical pen, which allows for free rotation of the electrosurgical pen to facilitate the operation of the electrosurgical pen.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A rotation structure of an electrosurgical pen, comprising a pen body and a cutting tip and a smoke suction tube mounted to the pen body, a rotation assembly coupled to the pen body, and a conduit connected to the rotation assembly, wherein the cutting tip is mounted on a top of the pen body and projects outwards from a front end of the pen body and is connected, via a conductive plate, to a circuit board to be further connected by the circuit to an electrical cable and the electrical cable is received in and extends through the smoke suction tube and the conduit, the smoke suction tube comprising an inner tube arranged such that the inner tube is exposed outside and projects frontwards from the front end of the pen body to be extendible and retractable with respect to out of the smoke suction tube to accommodate the cutting tip, the electrical cable being adapted to connect to an electrical power device, the conduit being adapted to connect to a smoke exhausting device, whereby when the electrosurgical pen is used in a surgical operation, the cutting tip is usable to conduct an electrosurgical operation and the smoke suction tube sucks and exhausts smoke generated during the electrosurgical operation, characterized in that:
the pen body has an end on which a rotary joint part is formed, the rotation assembly comprising inner and outer annular members that are rotatable with respect to each other, the rotation assembly being fit over and coupled to the rotary joint part of the pen body, the conduit being fit over and coupled to an outer circumference of the outer annular member of the rotation assembly, whereby in the use of the electrosurgical pen, the rotation assembly allows the smoke suction tube and the conduit to rotate with respect to each other so as to allow for free rotation of the electrosurgical pen to facilitate the operation of the electrosurgical pen.

2. The rotation structure of the electrosurgical pen according to claim 1, wherein the rotary joint part of the pen body comprises a flange formed on the end of the pen body, an insertion section extending from the flange, and a diameter-expanded retention section extending from the insertion section to receive the rotation assembly to be fit over the insertion section and retained in position by the retention section so as to prevent detachment of the rotation assembly.

3. The rotation structure of the electrosurgical pen according to claim 1, wherein the insertion section and the retention section of the rotary joint part of the end of the pen body comprise a plurality of slits formed therein to provide elasticity that facilitates fitting of the rotation assembly.

4. The rotation structure of the electrosurgical pen according to claim 1, wherein the rotation assembly comprises a bearing.
